(19) United States
(12) Patent Application Publication
(10) Pub. No.: US 2011/0270050 A1
(43) Pub. Date: Nov. 3, 2011

(54) Self Administered Health Assessment Method and Apparatus

(76) Inventors: Morteza Naghavi, Houston, TX (US); Albert Andrew Yen, Pearland, TX (US); Haider Ali Hassan, Houston, TX (US); David S. Panthagani, Houston, TX (US)

(21) Appl. No.: 13/099,536

(22) Filed: May 3, 2011

Related U.S. Application Data
(60) Provisional application No. 61/343,645, filed on May 3, 2010.

(51) Int. Cl. A61B 5/00

(52) U.S. Cl. 600/301

(57) ABSTRACT

The disclosure pertains to automated and subject operated medical diagnostic equipment that may be remotely monitored by trained medical personnel. The diagnostic equipment may be installed in publicly accessible locations such as airports or hotels. The diagnostic equipment may be located in a subject's home. The diagnostic equipment may be utilized by a subject as prompted by the diagnostic equipment. The prompts may be communicated audibly or by text. The text may be communicated using display screens. The display screens may be touch screens allowing the subject to answer audible or text questions. The diagnostic equipment may include a key pad either as a separate component or as part of a display screen. The subject may also respond verbally. The diagnostic equipment will be in communication with a computer processor containing software allowing the equipment to respond to subject verbal input including questions. This computer capability may be incorporated into the diagnostic equipment or remotely located accessed by means such as telemetry or the Internet.

### RELATED APPLICATION

This Application claims the benefit of and priority to Provisional Application Ser. No. 61/343,645 entitled Self-administered Health Assessment Method and Apparatus filed May 3, 2010. Provisional Application Ser. No. 61/343,645 is incorporated herein by reference in its entirety.

### BACKGROUND OF INVENTION

1. Field of Use

This disclosure pertains to automated and subject operated medical diagnostic equipment that may be remotely monitored by trained medical personnel.

2. Prior Art

Automated medical devices such as blood pressure monitors are known.

### SUMMARY OF DISCLOSURE

The disclosure pertains to automated and subject operated medical diagnostic equipment that may be remotely monitored by trained medical personnel. The diagnostic equipment may be installed in publicly accessible locations such as airports or hotels.

The diagnostic equipment may be utilized by a subject as prompted by the diagnostic equipment. The prompts may be communicated audibly or by text. The text may be communicated using display screens. The display screens may be touch screens allowing the subject to answer audible or text questions. The diagnostic equipment may include a key pad either as a separate component or as part of a display screen. The subject may also respond verbally. The diagnostic equipment will be in communication with a computer processor, containing software allowing the equipment to select from multiple possible responses based upon the subject's verbal or text input including questions. This capability is referred hereinafter as “intelligent diagnostic equipment”. This computer capability may be incorporated into the diagnostic equipment or remotely located accessed by means such as telemetry or the Internet.

The diagnostic equipment may be remotely monitored via video or audio facilities. The remote monitoring may include trained medical personnel. It will be appreciated that the computer processor communicating with the subject may summon trained medical personnel to the subject.

A subject may commence use of the diagnostic equipment through swiping or entering a credit card number. In another embodiment, the subject may place a thumb or finger tip on a scanner to establish identity. The information may identify the subject and allow access to a remote database, including a database containing the subject's medical information. In another embodiment, the subject may swipe or enter a personal health record (PHR). The PHR card may contain subject's medical information or allow access to a remote database. In another embodiment, the credit card provides a billing mechanism for the use of the diagnostic equipment.

As stated, the diagnostic equipment may have the capability to communicate with the subject. This may utilize computer processing capability as a component to the diagnostic equipment or may be remotely located. This may also involve a computer server in communication with the Cloud, an on-demand network accessed to a shared pool of configurable computing resources (e.g., networks, servers, storage, applications, and services) that can be rapidly provisioned and release with minimal management effort or service provider interaction. The Cloud network will include health assessment guidelines.

The diagnostic equipment may obtain information through text or audio communication. In another embodiment, the remotely stationed trained medical personnel may communicate directly with the subject. The medical personnel may have visual access to the subject. The medical personnel also may have access to the subject input and diagnostic equipment test results. The medical personnel may guide or instruct the subject on the correct procedure for use of the equipment to obtain optimal test results.

The diagnostic equipment may include, but is not limited to;
Card reader for card containing subject identifier and personal health record (capable of reading a magnetic strip or chip contained on the card);
Subject temperature monitor (e.g., thermometer);
Subject weight scale;
Blood pressure measuring component;
Ultrasound imaging component; (In one embodiment, the ultra sound imaging is capable of carotid artery imaging.)
Electro-cardiogram component;
Pulse oximeter;
Impedance monitoring to measure percent body fluid, percent body fat, and other aspects of body composition.
Speakers, camera, microphone, computer processor and server with Internet connectivity.

### SUMMARY OF DRAWINGS

The accompanying drawings, which are incorporated in and constitute a part of the specification, illustrate preferred embodiments of the invention. These drawings, together with the general description of the invention given above and the detailed description of the preferred embodiments given below, serve to explain the principles of the invention.

FIG. 1 illustrates one embodiment of the disclosure comprising a chair containing a plurality of automated or subject operated components that provide health related information regarding the subject.

### DETAILED DESCRIPTION OF DISCLOSURE

The present disclosure describes a device that combines automated or subject operated multiple function medical diagnostic equipment (hereinafter “diagnostic equipment” into a single booth or station which will allow a subject to retrieve or collect personal, individualized medical data that can be analyzed by the diagnostic equipment. The disclosure is intended to operate with minimal intervention by medical personnel. In one embodiment the medical personnel tele-support may be part of a home security surveillance service.

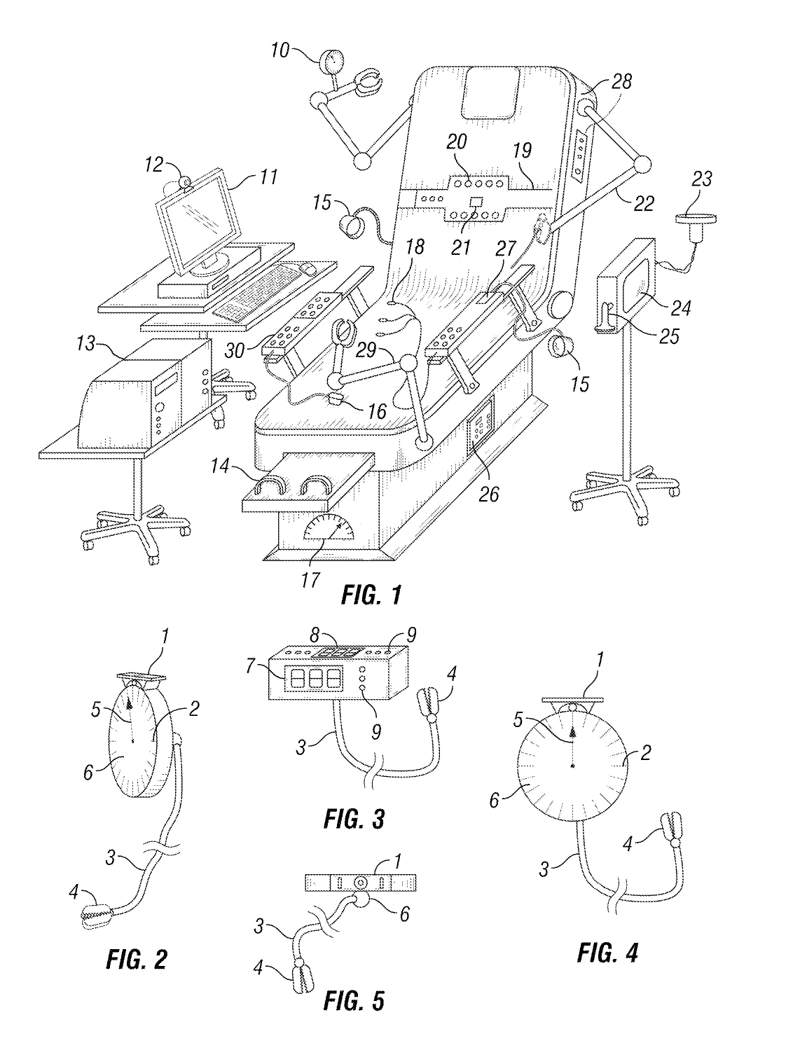

FIG. 1 illustrates an embodiment of the automated medical diagnostic apparatus. Illustrated is a reclining chair/bed 28. Also illustrated is a probe angle and level measurement device 10, a computer display 11, high definition camera for skin cancer and remote observation 12, PC and other medical device hardware housing 13, pneumatic cuffs to obtain ankle blood pressure 14, pneumatic cuffs to obtain signal from the arm 15, and pulse oximetry or photoplethysmography sensor 16. Also illustrated weight sensor 17, temperature probes 18, thoracic belt with EKG electrodes, PCG, Chest sound sensor, impedance, and other sensors 19, EKG and impedance sensors 20, PCG and chests sound sensor 21, nose mouth gas analysis mask 22, ultrasound probe for AAA screening 23, ultrasound machine 24 and ultrasound probe for carotid and femoral screening 25. Further illustrated are biomedical devices 26, point of care blood sample testing machine (blood chemical analyzer) 27, ultrasound probe holder for AAA screening and Doppler flow measurements 29 and computer processor and device control panel 30.

FIGS. 2 through 5 illustrate a bubble indicator to achieve horizontal leveling 1, graduated scale for tilt angle measurement 2, flexible cable to change/adjust position in three dimensions 3, alligator clip to attach the device 4, angle indicator needle 5, customized bezel for ultrasound angle measurement 6, digital readout display for vertical angle 7, digital readout display for horizontal angle 8, and visual cues for leveling 9.

The subject can be guided through the test procedures and data collection protocol by audio or visual commands and instructions. The diagnostic equipment can also ask questions and record responses. These responses can be recorded in digital format. The diagnostic equipment can also ask follow-on questions based upon the subject's response to a prior question. (Machines having this capability are used in telephone call systems.) The diagnostic equipment of the present disclosure may also utilize visual inputs in exchanges with the subject. These may be graphics or written information. The equipment may provide pre-recorded or remotely transmitted audio visual guidance, asking questions and retrieving answers through a computer controlled system, and analyzing the input information utilizing computer software programmed to correlate and assess health related subject responses and test data. This combination of inputs and responses will be referred throughout this disclosure as “intelligent diagnostic equipment” or “computer instructions”. For example, the temperature monitor recording a subject's temperature above 101° F. may activate a diagnostic equipment protocol including questions or instructions to the subject. The temperature monitor recording a temperature above 103° F. may summon intervention by trained medical personal.

It is also intended that the diagnostic equipment be monitored from a remote location by trained medical personnel. This monitoring can be via audio or visual equipment (hereinafter “tele-support”). Each medical personnel may monitor a plurality of diagnostic equipment units. It is intended that the remotely located medical personnel will have access to the subject's stored health information and the test results of the diagnostic equipment. The trained medical personnel may cause the diagnostic equipment to re-perform test procedure and have real time access to the test results. The remotely located medical personnel may have communication with the subject. The process is hereinafter termed “tele-medicine”.

In a preferred embodiment, the diagnostic equipment includes:
a) Card reader (capable of reading a magnetic strip or chip contained on the card);
b) Subject temperature monitor (e.g., thermometer);
c) Subject weight scale;
d) Blood pressure measuring component;
e) Ultrasound imaging component; (In one embodiment, the ultra sound imaging is capable of carotid artery imaging.)
f) Electro-cardiogram component;
g) Pulse oximeter; h) Impedance monitoring to measure percent body fat.
h) Speakers, camera, microphone, computer processor and server with Internet connectivity.
i) Speakers, camera, microphone, server with Internet connectivity, and computer processor programmed to receive card identifier information of the subject, receive subject medical information from third source, e.g., personal health records via the Internet, instruct the subject on operation of the diagnostic equipment, receive and respond to subject's questions regarding equipment operation, receive diagnostic equipment results (either directly from equipment of inputted by subject), evaluate the result with programmed values or algorithms or subject's received medical information, evaluate the information to determine if tele-support medical personnel should be summoned, record diagnostic equipment results, communicate medical information results to subject, respond to subject's question using programmed database modified to reflect diagnostic equipment results or evaluate and determine whether to summon tele-support medical personnel to respond to subject's question, provide instructions to subject regarding additional test or ask subject if he/she wants to perform additional test, communicate diagnostic equipment results to tele-support personnel or subject's medical information database, printout medical information results, and reset diagnostic equipment for next subject. The computer processor may also be programmed to receive input from the tele-support medical personnel including directions on operating the diagnostic equipment.

In another embodiment, the diagnostic equipment would also include a blood chemical analyzer;

In one embodiment, the subject may use the diagnostic equipment to take anthropometric measurements, including but not limited to:

a) BMI (body mass index)

b) Weight

c) Height

d) Waist circumference

e) Body fat (composition) measurements using impedance signals

The measured data, e.g., waist circumference and subject height, may be entered into the diagnostic equipment and data base. Subject height may be measured using a camera viewing the subject standing proximate to a vertical measuring scale.

In one embodiment, the diagnostic equipment contains ultrasound image component that may be used to measure Carotid Intima-Media Thickness. This can be used to assess subclinical atherosclerosis.

The diagnostic equipment may, in one embodiment, be equipped with the components to allow the subject to conduct a cardiovascular disease screening comprising data input, including but not limited to:
a) Electrocardiography (belt)
b) Abdominal aortic aneurysm screening
c) Carotid IMT (intima media thickness) and plaque screening
d) Vascular reactivity measurement, including but not limited to,
   i) Contralateral vascular reactivity
   ii) Digital thermal reactivity
e) Endothelial function measurement, including but not limited to, peripheral arterial tonometry (pressure)
f) Doppler ultrasound parameters, including but not limited to,
   i) Pulse wave velocity
   ii) Pulse wave flow
   iii) Pulse transit time
   iv) Doppler flow velocity
g) Plaque screening and characterization
h) Ankle-brachial index
   i) Toe-brachial Index (test of the large toe systolic pressure with brachial artery systolic pressure)
   ii) Toe-finger Index
i) Blood pressure and heart rate measurement
j) Impedance Cardiograph (ICG) signals and calculated parameters
k) Photoplethysmography (PPG) signals and calculated parameters
l) Pulse oximetry

The device may include apparatus for collection of blood samples. This can include tourniquets to facilitate collection of arterial blood and “finger prick” devices for collecting additional blood samples. The diagnostic equipment permits the subject to collect a sample of his/her blood. In one embodiment, blood testing may comprise one or more of the following tests:
a) Lipid panel
b) High Sensitivity C-Reactive Protein (hsCRP) Assay
c) HbA1c (a test that measures the amount of glycated hemoglobin in the subject's blood)
d) Liver enzymes
e) Blood thrombogenicity (Thrombogenicity refers to the tendency of a material in contact with the blood to produce a thrombus, or clot. It not only refers to fixed thrombi but also to emboli, thrombi which have become detached and travel through the bloodstream. Thrombogenicity can also encompass events such as the activation of immune pathways and the complement system.)
f) Glucose
g) Natriuretic factors (Natriuretic peptides are a family of structurally related but genetically distinct hormones/paracrine factors that regulate blood volume, blood pressure, ventricular hypertrophy, pulmonary hypertension, fat metabolism, and long bone growth. The mammalian members are atrial natriuretic peptide, B-type natriuretic peptide, C-type natriuretic peptide, and possibly osteocrin/musclin. Three single membrane-spanning natriuretic peptide receptors (NPRs) have been identified. Two, NPR-A/GC-A/NPR1 and NPR-B/GC-B/NPR2, are transmembrane guanylyl cyclases, enzymes that catalyze the synthesis of cGMP.)
h) Troponin levels (Troponin is a complex of three regulatory proteins that is integral to muscle contraction in skeletal and cardiac muscle, but not smooth muscle. Discussions of troponin often pertain to its functional characteristics and/or to its usefulness as a diagnostic marker for various heart disorders.)
i) Proteomic blood chemistry
j) Genomic blood chemistry

In a further embodiment of the disclosure, the subject may input data from subject controlled diagnostic equipment comprising at least one of the following devices;
a. Lymph node inflammation detector (palpography)
b. Breast cancer nodules detector
c. Lung/breathing sound and percussion detector.
d. Body temperature detector.

In one embodiment, the device may permit machine assessment of skin cancer using a camera system. The device will be equipped with at least one high resolution digital cameral that can photograph the subject's whole body surface area. This can include the anterior and posterior of the body surface area. The digital photographs can be scan for skin surface anomalies that may require further evaluation for cancer or cancer precursor sites. The photographs may also be evaluated for sores or infection.

In another embodiment, the device may utilize a high resolution digital camera to photograph the subject's mouth for potential abnormalities including but not limited to cancer, sores or infection.

The subject conducting a spirometry analysis using the diagnostic equipment, i.e., measuring lung function, specifically the measurement of the amount (volume) and/or speed (flow) of air that can be inhaled and exhaled. Spirometry is an important tool used for generating pneumotachographs which are helpful in assessing conditions such as asthma, pulmonary fibrosis, cystic fibrosis, and COPD.

Tele-support is monitored support from a remote location. Multiple diagnostics can be monitored from a single tele-support person. The tele-support person will be a skilled medical professional. There may be an operator at the location of the diagnostic equipment to assist the subject in the functioning of the equipment.

Depending upon the test results, commercial information can be given (video) to the subject. This may be medical information relevant to the test results obtained by the diagnostic equipment or the subject's PHR. This information can be delivered through the diagnostic equipment, e.g., monitor screens.

The diagnostic equipment is intelligent, i.e., gives instructions, receives information, processes responses to questions, etc. The diagnostic equipment further provides pre-recorded or remotely transmitted audio visual guidance, asking questions and retrieving answers through a computer controlled system, and analyzing the input information utilizing computer software programmed to correlate and assess health related subject responses and test data. This information collection process and capability is hereinafter referred to as “equipment questions”

The medical personnel tele-support may access the diagnostic equipment through an onsite camera. Diagnostic equipment can be placed is any public accessible space, i.e., apartment buildings, airports, hotels, schools, universities, pharmacies. The diagnostic equipment can be individually located in a secure private area.

This specification is to be construed as illustrative only and is for the purpose of teaching those skilled in the art the manner of carrying out the invention. It is to be understood that the forms of the invention herein shown and described are to be taken as the presently preferred embodiments. As already stated, various changes may be made in the shape, size and arrangement of components or adjustments made in the steps of the method without departing from the scope of this invention. For example, equivalent elements may be substituted for those illustrated and described herein and certain features of the invention maybe utilized independently of the use of other features, all as would be apparent to one skilled in the art after having the benefit of this description of the invention.

While specific embodiments have been illustrated and described, numerous modifications are possible without departing from the spirit of the invention, and the scope of protection is only limited by the scope of the accompanying claims.

What we claim is:

1. A method for a subject to conduct self-administered health assessment by acquiring multiple health related signals and health related information assisted by diagnostic equipment instructions onsite, a cloud computing service, and a medical personnel tele-support.

2. The method of claim 1 further comprising the medical personnel tele-support to be part of a home security surveillance service.

3. The said method of claim 1 further utilizing equipment instructions comprising pre-recorded or remotely transmitted audio visual guidance, diagnostic equipment asking questions and retrieving subject answers through a computer controlled system, and analyzing the answers and diagnostic equipment data utilizing computer software or cloud computing services programmed to correlate and assess health related subject responses and test data.

4. The said method of claim 1 for a subject conducting a cardiovascular disease screening comprising four or more of the following data inputs, including but not limited to:
a) conducting electrocardiography
b) conducting abdominal aortic aneurysm screening
c) conducting carotid IMT and plaque screening
d) measuring vascular function measurement, including but not limited to,
i) Contralateral vascular reactivity
ii) Digital thermal reactivity
e) conducting plaque screening and characterization
f) conducting ankle-brachial index
i. Toe-brachial Index
ii. Toe-finger Index
g) measuring blood pressure and heart rate
h) measuring impedance cardiograph (ICG) signals and calculated parameters
i) measuring photoplethysmography (PPG) signals and calculated parameters
j) measuring pulse oximetry

5. A diagnostic system comprising automated medical diagnostic test or measurement components that are self administered by a subject and further comprising audio and visual instructional capability to allow a subject to conduct self-administered health assessment by acquiring multiple health related signals and information and supported by both cloud computing and human tele-support.

6. The system of claim 5 capable of performing health assessments further comprising:
a) Card reader for card containing subject identifier and personal health record (capable of reading a magnetic strip or chip contained on the card);
b) Subject temperature monitor (e.g., thermometer);
c) Subject weight scale;
d) Blood pressure measuring component;
e) Ultrasound imaging component; (In one embodiment, the ultra sound imaging is capable of carotid artery imaging.)
f) Electro-cardiogram component;
g) Pulse oximeter;
h) Impedance monitoring to measure percent body fluid, percent body fat, and other aspects of body composition.
i) Speakers, camera, microphone, computer processor and server with Internet connectivity.

7. The system of claim 5 further comprising the following:
a) a blood chemical analyzer.
b) Lymph node tissue detector (palpography);
c) Breast cancer nodule detector;
d) Lung/breathing sound and percussion detector;
e) Regional body impedance measurement device.

8. The system of claim 5 further comprising an apparatus for conducting an eye examination wherein the eye examination data is machine recorded and correlated and analyzed by a computer, and including but not limited to:
a) Retinoscopy;
b) Glaucoma screening;
c) Corneal and retinal topography;
d) Refraction test;
e) Visual field test;
f) Visual acuity test;
g) Macular degeneration screening;
h) Cataracts screening (OCT, slide/lamp).

9. The system of claim 5 for subject self-administered cardiovascular disease screening, including but not limited to:
a) Electrocardiography;
b) Abdominal aortic aneurysm screening component;
c) Carotid IMT and plaque screening component;
d) Vascular reactivity measurement component, including but not limited to,
i) Contralateral vascular reactivity;
ii) Digital thermal reactivity;
